(19) United States
(12) Patent Application Publication
(10) Pub. No.: US 2007/0173535 A1
(43) Pub. Date: Jul. 26, 2007

(54) Oxadiazoles having antiproliferative activity

(76) Inventors: Giorgio Cignarella, Milano (IT); Gerardo Pinna, Sassari (IT); Gabriele Murineddu, Sassari (IT)

Correspondence Address: YOUNG & THOMPSON, 745 SOUTH 23RD STREET, 2ND FLOOR, ARLINGTON, VA, 22202, US

(21) Appl. No.: 11/596,392

(22) PCT Filed: Jan. 31, 2003
(86) PCT No.: PCT/IB03/00322

§ 371 Date: Feb. 15, 2007

(30) Foreign Application Priority Data
Feb. 8, 2002 (IT) MI2002A000236

(51) Int. Cl. A61K 31/4245; C07D 209/56

(52) U.S. Cl. 514/364; 548/143; 548/427

(57) ABSTRACT

Oxadiazole derivatives of general formula (1) in which X, Y, R and R2 have the meanings defined in the disclosure. The compounds have antiproliferative activity against a number of human tumors cell lines and can therefore be used for the preparation of antitumor medicaments.

The present invention relates to oxadiazole derivatives of general formula (I)

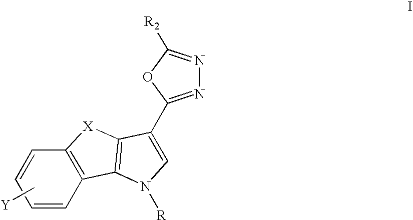

in which:

X is a CH₂, CH₂CH₂ or CH═CH group;

R is hydrogen, straight or branched C₁-C₄ alkyl, or phenyl-C₁-C₂-alkyl;

R₂ is hydrogen, straight or branched C₁-C₄ alkyl, or phenyl, optionally substituted with one more groups, which can be the same or different, selected from halogen (fluorine, chlorine, bromine, iodine), straight or branched C₁-C₄ alkyl, C₁-C₃ alkoxy, trifluoromethyl;

Y is hydrogen, halogen (fluorine, chlorine, bromine, iodine), straight or branched C₁-C₄ alkyl, C₁-C₃ alkoxy, trifluoromethyl.

C₁-C₄ Alkyl is preferably methyl or ethyl.

Phenyl-C₁-C₂-alkyl is preferably benzyl, optionally substituted on the phenyl ring with one more groups, which can be the same or different, selected from halogen (fluorine, chlorine, bromine, iodine), straight or branched C₁-C₃ alkyl, C₁-C₃ alkoxy, trifluoromethyl.

C₁-C₃ Alkoxy is preferably methoxy or ethoxy.

The invention also relates to the non toxic salts and solvates of compounds (I).

In compounds of formula (I), X is preferably a CH₂ or a CH═CH group, R is hydrogen or methyl and R₂ is hydrogen, methyl or optionally substituted substituted phenyl, as defined above.

More preferably, R₂ is phenyl or substituted phenyl, as defined above.

Examples of preferred compounds according to the invention comprise:
3-[[1,3,4]oxadiazol-2′-yl]-1,4-dihydro-indeno[1,2-b]pyrrole;
3-[[1,3,4]oxadiazole-5′-methyl-2′-yl]-1,4-dihydro-indeno[1,2-b]pyrrole;
3-[[1,3,4]oxadiazole-5′-phenyl-2′-yl]-1,4-dihydro-indeno[1,2-b]pyrrole;
1-methyl-3-[[1,3,4]oxadiazol-2′-yl]-1,4-dihydro-indeno[1,2-b]pyrrole;
3-[[1,3,4]oxadiazol-2′-yl]-4,5-dihydro-1H-benzo[g]indole;
3-[[1,3,4]oxadiazole-5′-methyl-2′-yl]-4,5-dihydro-1H-benzo[g]indole;
3-[[1,3,4]oxadiazole-5′-phenyl-2′-yl]-4,5-dihydro-1H-benzo[g]indole;
1-methyl-3-[[1,3,4]oxadiazol-2′-yl]-1H-benzo[g]indole;
3-[[1,3,4]oxadiazol-2′-yl]-1H-benzo[g]indole;
3-[[1,3,4]oxadiazole-5′-methyl-2′-yl]-1H-benzo[g]indole;
3-[[1,3,4]oxadiazole-5′-phenyl-2′-yl]-1H-benzo[g]indole.

Compounds of formula (I) have cytotoxic activity and can be used as antitumor and antiproliferative medicaments.

Compounds of formula (I) can be prepared by reacting hydrazides of formula (II) (scheme 1) wherein X, Y and R are as defined above, with an orthoformate of formula (III),
R₂C(OEt)₃ (III)

in which R₂ has the meaning defined above.

The reaction is carried out in a dipolar aprotic solvent, preferably dimethylformamide.

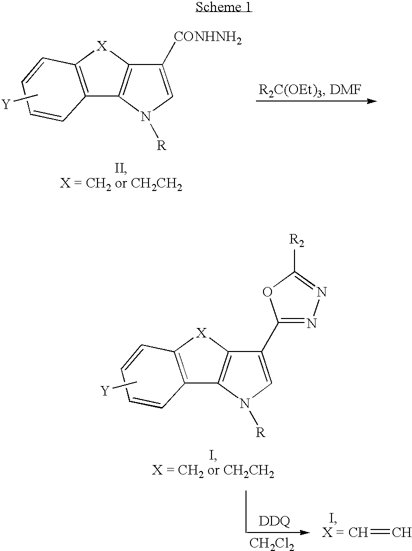

Compounds (I) in which X is CH═CH can be obtained by treatment of a compound of formula (I), in which X is CH₂CH₂, with DDQ in CH₂Cl₂.

Compounds of formula (II), which are novel and are a further object of the invention, can be obtained as shown in Scheme 2 (step i), starting from a compound of formula (IV), in which X is CH₂ or CH₂CH₂ and Y is as defined above, by reaction with a cyanoacetic acid ester of formula V
CNCH₂COOR₁ (V)

in which R₁ has the same meanings as R defined above except for hydrogen, to give the compounds of formula VI, which are then treated with hydrochloric acid gas in ether solution (step ii), to yield the compounds of formula (VII) in which R is hydrogen. The reaction which affords compounds (VI) is also accompanied by formation of compounds (VIII), which are removed with conventional techniques. Step i) is preferably carried out in acetone in the presence of potassium bicarbonate, but also other solvents and bases may be used. Compounds (VII) can be optionally alkylated at the pyrrole nitrogen (step iii) with an alkylsulfonate of formula (IX).
R₂SO₄ (IX)

in which R has the meanings defined above except for hydrogen.

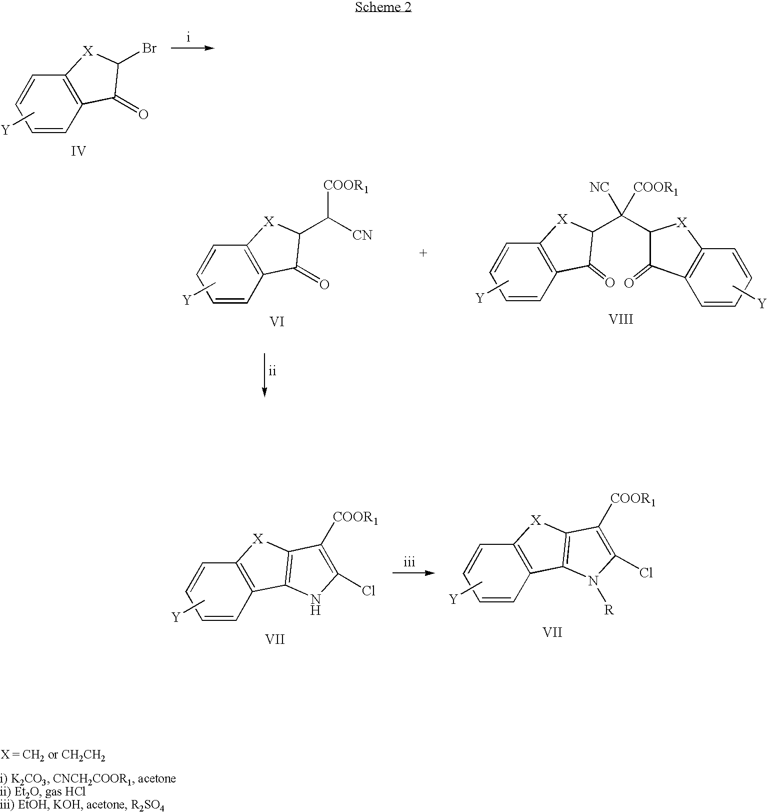

Reductive dehalogenation of compounds (VII) with ammonium formate in methanol and Pd—C yields compounds (X), as shown in Scheme 3. The latter are treated with DDQ in CH₂CL₂ to obtain compounds (X) in which X is CH═CH.

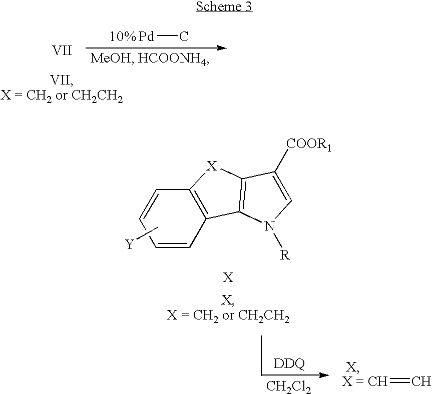

Compounds (X) are then reacted with hydrazine (scheme 4) to give compounds of formula (II).

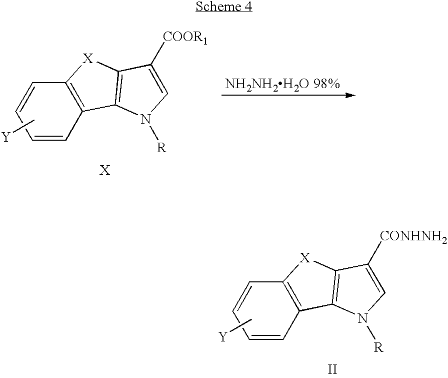

The cytotoxic activity of compounds (I) was evidenced in primary screening studies on 60 human tumor cell lines. Compounds (I) showed marked antiproliferative activity, in particular against leukemias, colon and breast tumors.

Compounds of formula (I) may therefore be used as antitumor medicaments, more particularly for the treatment of leukemias, colon and breast tumors. For this purpose, compounds of formula (I) will be formulated in admixture with suitable excipients and/or carriers according to conventional techniques. The pharmaceutical compositions of the invention may be administered through the oral, parenteral, rectal or topical route.

The therapeutical dosages will depend on a number of factors, such as the severity of the disease to treat, the weight, sex and age of the patient as well as the pharmaco-toxicological and pharmacokynetics characteristics of the selected compound of formula (I). In principle, the daily dosage will range from 0.01 to 10 mg/kg body weight of the patient.

The invention is illustrated in greater detail by the following examples.

### EXAMPLES

Materials and Methods

Compound (IVa) (α-bromoindan-1-one) is commercially available (Aldrich Chemical Co.), whereas compound (IVb) (α-bromo-tetral-1-one) is synthesized according literature [Wilds A. L., Johnson J. A. Jr., J. Am. Chem. Soc., 68, 86-89 (1946)].

Flash chromatography was performed on silica gel Merck 60 (230-400 mesh ASTM). Thin layer chromatography (TLC) was carried out on plates (0.2 mm) Polygram®SIL N—HR-/HV₂₅₄.

Melting points were determined with a Thomas-Hoover melting point capillary apparatus and are not corrected.

IR spectra were recorded on NaCl pellets (on thin films of the products in Nujol) with a Perkin Elmer 781 IR spectrophotometer and are expressed in ν cm⁻¹.

NMR spectra were recorded with an XL-200 Varian instrumentation at 200 MHz and chemical shifts are expressed in ppm.

### Example 1

### Ethyl 2-cyano-2(1-oxy-2,3-dihydro-1H-inden-2-yl)acetate (VIa)

A solution of α-bromoindan-1-one (IVa) (5.02 mmol) in acetone (10.9 ml) is added, drop by drop, with a suspension of CNCH₂COOEt (40 mmol) and K₂CO₃ (10 mmol) at 40-45° C. The reaction mixture is stirred at 40-45° C. for 1 h and then cooled to room temperature. Ethyl acetate (10 ml) and water (10 ml) are added under stirring; the organic layer is separated, washed with a 10% KH₂PO₄ solution (7.5 ml) and ice (5 ml), dried over Na₂SO₄ and concentrated under reduced pressure to give a crude oil, which is subsequently distilled (150° C./1 mmHg) and purified by flash chromatography (eluent: petroleum ether/ethyl acetate 8:2)

Compound (VIb) is prepared following the same procedure.

### Example 2

### Ethyl 1,4-dihydro-indeno[1,2-b]pyrrole-2-chloro-3-carboxylate (VIIa)

A solution of compound (VIa) (6.0 mmol) in ethyl ether (15 ml), cooled in ice at 0-5° C., is added with hydrochloric acid gas (1.86 g, 5.1 mmol). The solution is kept under stirring at room temperature for 24 h, then ethyl ether and hydrochloric acid excess are removed with nitrogen. The solid residue is triturated in methanol to give cream crystals.

Compound (VIIb) is prepared following the same procedure, but preparing the starting solution with 30 ml of ethyl ether.

### Example 3

### Ethyl 1-methyl-1,4-dihydro-indeno[1,2-b]pyrrole-2-chloro-3-carboxylate (VIIc)

0.28 g (4.99 mmol) of KOH are dissolved in a solution of compound (VIIa) (4.14 mmol) in ETOH (23 ml); the solvent is evaporated off under reduced pressure and the residue is dissolved in acetone (18.6 ml), then added with 0.78 ml (8.28 mmol) of Me₂SO₄. The mixture is kept under stirring at room temperature until completion of the reaction (about 30 min, TLC petroleum ether/ethyl acetate 8/2). The precipitated solid is filtered off and the solution is concentrated under reduced pressure to give an oil which solidifies upon standing.

Compound (VIb) is prepared following the same procedure, starting from compound (VIId).

### Example 4

### Ethyl 1,4-dihydro-indeno[1,2-b]pyrrole-3-carboxylate (Xa)

A solution of 2.72 mmol of chloroester (VIIa) and 13.6 mmol (0.86 g) of ammonium formate in 22.44 ml of methanol is added with 0.18 g of 10% Pd—C. The mixture is kept under nitrogen stream and under stirring until completion of the reaction (about 4 hours, TLC: petroleum ether/ethyl acetate 8/2), after that is filtered and the solution is evaporated under reduced pressure.

The resulting residue is taken up into water; the resulting suspension is filtered and the precipitate is washed with ethanol.

Compound (Xb) is prepared following the same procedure. Compounds (Xc) and (Xd) are purified by extraction of the evaporation residue aqueous solution with ether, evaporation of the ether phase and distillation under low pressure.

### Example 5

### Ethyl 1-methyl-1H-benzo[g]indole-3-carboxylate (Xf)

Compound (Xd) (0.67 g, 2.66 mmol) is dissolved in 10 ml of CH₂Cl₂, 1.81 g (7.98 mmol) of DDQ are added thereto and the mixture is left under stirring at room temperature for 5 min, then evaporated and the residue is purified by flash chromatography eluting with petroleum ether/ethyl acetate 8/2.

Following the same procedure, starting from compound (Xb), compound (Xe) is prepared.

### Example 6

### 1,4-Dihydro-indeno[1,2-b]pyrrole-3-carbohydrazide (IIa)

A mixture of 2 mmol of ester (Xa) and 1.94 ml (40 mmol) of hydrazine is refluxed until completion of the reaction (about 30 min., TLC: CHCl₃/MeOH 9/1), then left to cool and poured into ice. The precipitate is filtered, washed with water and dried in the air.

Following the same procedure, starting from the esters (Xb, c, f), hydrazides (IIb, c, f) are prepared.

### Example 7

### 3-[[1,3,4]Oxadiazol-2′-yl]-1,4-dihydro-indeno[1,2-b]pyrrole (Ia)

A solution of 2.48 mmol of hydrazide (IIa) and 2.73 mmol of ethyl orthoformate in 5 ml of DMF is refluxed for about 8 h (TLC: CHCl₃/MeOH 9/1), then left to cool and poured onto ice. The precipitate is filtered, washed with water and dried in the air.

Following the same procedure, using ethyl orthoacetate or orthobenzoate, compounds (Ia-g, m) are prepared, whereas compounds (Ih, i, l) are prepared from compounds (Ie, f, g) following the procedure of Example 4.

TABLE 1

| 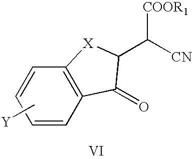 | Yield | M.p. | IR | ¹H-NMR(CDCl₃) |
|---|---|---|---|---|
| VIa | 72.30% | 58-60° C. | 1715(C═O), | 1.21(t, 3H, CH₃); 2.58-2.59(dd, |
| (X = CH₂, Y = H, R₁ = Et) | | | 1745(C═O), | 2H, CH₂); 2.93-3.03(dd, 1H, CH); |
| | | | 2250(CN) | 3.40-3.52(dd, 1H, CH); 4.11(q, |
| | | | | 2H, CH₂); 7.38(t, 1H, CH); 7.46 |
| | | | | (d, 1H, CH); 7.60(t, 1H, CH); 7.77 |
| | | | | (d, 1H, CH) |
| VIb | 96.5% | 54-56° C. | 1680(C═O), | 1.37(t, 3H, CH₃); 2.36(q, 2H, |
| (X = CH₂CH₂, Y = H, | | | 1740(C═O), | CH₂); 3.12(t, 2H, CH₂); 4.33(q, |
| R₁ = Et) | | | 2250(CN) | 2H, CH₂); 4.36(q, 1H, CH); 4.45 |
| | | | | (d, 1H, CH); 7.30(d, 1H, CH); |
| | | | | 7.34(t, 1H, CH); 7.54(t, 1H, CH); |
| | | | | 8.06(d, 1H, CH) |

TABLE 2

| 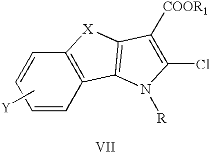 | Yield | M.p. | IR | ¹H-NMR(CDCl₃) |
|---|---|---|---|---|
| VIIa | 69.8% | 206-208° C. | 1670(C═O), | 1.40(t, 3H, CH₃), 3.66(s, 2H, |
| (X = CH₂, Y = H, R₁ = Et, | | | 3240(NH) | CH₂), 4.35(q, 2H, CH₂), 7.11(t, |
| R = H) | | | | 1H, CH), 7.25(t, 1H, CH), 7.42 |
| | | | | (d, 1H, CH), 7.47(d, 1H, CH), |
| | | | | 11.58(br s, 1H, NH exchanges |
| | | | | with D₂O) |
| VIIb | 43.0% | 185-186° C | 1660(C═O), | 1.38(t, 3H, CH₃), 2.88-3.05(m, |
| (X = CH₂CH₂, Y = H, | | | 3210(NH) | 4H, CH₂x2), 4.35(q, 2H, CH₂), |
| R₁ = Et, R = H) | | | | 7.10-7.26(m, 4H, Ph), 8.78(br s |
| | | | | 1H, NH exchanges with D₂O) |
| VIIc | 95.2% | 102-104° C. | 1700(C═O) | 1.39(t, 3H, CH₃), 3.65(s, 2H, |
| (X = CH₂, Y = H, R₁ = Et, | | | | CH₂), 3.87(s, 3H, CH₃), 4.35(q, |
| R = CH₃) | | | | 2H, CH₂), 7.15(t, 1H, CH), 7.29 |
| | | | | (t, 1H, CH), 7.44(t, 1H, CH), |
| | | | | 7.50(d, 1H, CH) |
| VIId | 95.3% | 53-55° C. | 1700(C═O) | 1.38(t, 3H, CH₃), 2.82-2.98(m, |
| (X = CH₂CH₂, Y = H | | | | 4H, CH₂x2), 3.88(s, 3H, CH₃), |
| R₁ = Et, R = CH₃) | | | | 4.34(q, 2H, CH₂), 7.13-7.41(m, |
| | | | | 4H, CHx4) |

TABLE 3

| 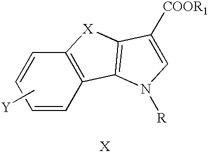 | Yeld | M.p./B.p. | IR | ¹H-NMR |
|---|---|---|---|---|
| Xa | 77.35% | 176-178° C. | 1670(C═O), | (CDCl₃) 1.39(t, 3H, CH₃); 3.70(s, |
| (X = CH₂, Y = H, R = H, | | | 3240(NH) | 2H, CH₂), 4.35(q, 2H, CH₂); 7.14 |
| R₁ = Et) | | | | (d, 1H, CH); 7.23(t, 1H, CH); 7.48 |
| | | | | (s, 1H, CH); 7.51(d, 1H, CH); |
| | | | | 8.82(br s, 1H, NH exchanges with |
| | | | | D₂O) |
| Xb | 71.70% | 140-142° C. | 1660(C═O), | (CDCl₃) 1.36(t, 3H, CH₃); 2.98(t, |
| (X = CH2CH2, Y = H, | | | 3250(NH) | 2H, CH₂); 3.04(t, 2H, CH₂); 4.32 |
| R = H, R1 = Et) | | | | (q, 2H, CH₂); 7.14-7.25(m, 4H, |
| | | | | CH); 7.43(s, 1H, CH); 8.65(br s, |
| | | | | 1H, NH exchanges with D₂O) |
| Xc | 81.90% | 120-121° C./ | 1695(C═O) | (CDCl₃) 1.37(t, 3H, CH₃); 3.66(s, |
| (X = CH₂, Y = H, | | 0.5 mm Hg | | 2H, CH₂); 3.93(s, 3H, CH₃); 4.32 |
| R = CH₃, R₁ = Et) | | | | (q, 2H, CH₂); 7.16(d, 1H, CH); |
| | | | | 7.27(s, 1H, CH); 7.30(t, 1H, CH); |
| | | | | 7.44(d, 1H, CH); 7.50(d, 1H, CH) |
| Xd | 70.58% | 59-60° C./ | 1700(C═O) | (CDCl3) 1.35(t, 3H, CH3); 2.86 |
| (CH₂CH₂, Y = H, | | 0.5 mm Hg | | (t, 2H, CH2); 2.98(t, 2H, CH2); |
| R = CH₃, R₁ = Et) | | | | 3.93(s, 3H, CH3); 4.34(q, 2H, |
| | | | | CH2); 7.10-7.30(m, 4H, CHx4); |
| | | | | 7.43(d, 1H, CH). |
| Xe | 56.41% | 144.5-145.5° C. | 1660(C═O), | (CDCl₃ + DMSO) 1.39(t, 3H, |
| (CH═CH, Y = H, R = H, | | | 3250(NH) | CH₃); 4.34(q, 2H, CH₂); 7.39-7.45 |
| R₁ = Et) | | | | (m, 2H, CHx2); 7.54(d, 1H, CH); |
| | | | | 7.85(d, 1H, CH, J_{AB}=8.8Hz); |
| | | | | 7.87-7.90(m, 1H, CH); 8.14(d, |
| | | | | 1H, CH); 8.31(d, 1H, CH, |
| | | | | J_{AB}=8.8Hz); 12.33(br s, |
| | | | | 1H, NH exchanges with |
| | | | | D₂O) |
| Xf | 87.88% | 116-118° C. | 1690(C═O) | (CDCl₃) 1.43(t, 3H, CH₃); 4.24(s, |
| (CH═CH, Y = H, | | | | 3H, CH₃); 4.41(q, 2H, CH₂); 7.45-7.54 |
| R = CH₃, R₁ = Et) | | | | (m, 2H, CHx2); 7.64(d, 1H, |
| | | | | CH, J_{AB}=8.2Hz); 7.70(s, 1H, |
| | | | | CH); 7.96(d, 1H, CH); 8.30(d, |
| | | | | 1H, CH, J_{AB}=8.2Hz); 8.40(d, 1H, |
| | | | | CH) |

TABLE 4

| 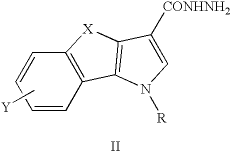 | Yeld | M.p. | IR | ¹H-NMR(CDCl₃ + DMSO) |
|---|---|---|---|---|
| IIa | 80.0% | 254-256° C. | 1650 C═O), | 3.65(s, 2H, CH₂); 7.06(t, 1H, |
| (X = CH₂, Y = H, R = H) | | | 3190-3270 | CH); 7.22(t, 1H, CH); 7.39-7.45 |
| | | | (NH₂) | (d, 2H, CHx2); 7.88(s, |
| | | | | 1H, CH); 8.68(br s, 1H, NH |
| | | | | exchanges with D₂O) |
| IIb | 80.0% | 268-270° C. | 1655 C═O), | 2.66(t, 2H, CH₂); 2.84(t, 2H, |
| (X = CH₂CH₂, Y = H, | | | 3140-3240 | CH₂); 4.30(br s, 2H, NH₂ che |
| R = H) | | | (NH₂), 3300 | scambia con D₂O); 6.66(s, |
| | | | (NH) | 1H, CH); 7.08(d, 1H, CH); |
| | | | | 7.17(t, 2H, CHx2); 7.80(d, |
| | | | | 1H, CH); 9.25(br s, 1H, NH |
| | | | | exchanges with D₂O); 11.68(br |
| | | | | s, 1H, NH exchanges with D₂O) |
| IIc | 88.03% | 219-221° C. | 1645 C═O), | 2.85(br s, 2H, NH₂ exchanges |
| (X = CH₂, Y = H, | | | 3300-3320 | with D₂O); 3.66(s, 2H, CH₂); |
| R = CH₃) | | | (NH₂) | 3.93(s, 3H, CH₃); 7.12(t, 1H, |
| | | | | CH); 7.24-7.34(m, 2H, |
| | | | | CHx2); 7.97(s, 1H, NH |
| | | | | exchanges with D₂O) |
| IIf | 58.1% | 216-218° C. | 1625 C═O); | 3.42(br s, 2H, NH₂ exchanges |
| (CH═CH, Y = H, | | | 3320(NH) | with D₂O); 4.30(s, 3H, CH₃); |
| R = CH₃) | | | | 7.45-7.55(m, 2H, CHx2); 7.57 |
| | | | | (d, 1H, CH, J_{AB}=8.4Hz); 7.84 |
| | | | | (s, 1H, CH); 7.96(d, 1H, CH); |
| | | | | 8.37(d, 1H, CH, J_{AB}=8.4 Hz); |
| | | | | 9.13(br s, 1H, NH exchanges |
| | | | | with D₂O) |

TABLE 5

| 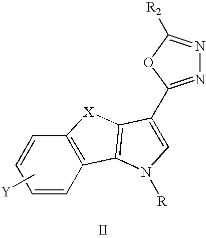 | Yeld | M.p. | IR | ¹H-NMR |
|---|---|---|---|---|
| Ia | 75.0% | 275-278° C. | 1605(C═N), | (DMSO) 3.68(s, 2H, CH₂); 7.14(t, 1H, |
| (X = CH₂, Y = H, | | (EtOH) | 3180(NH) | CH); 7.31(t, 1H, CH); 7.51(d, 1H, |
| R = H, R₂ = H) | | | | CH); 7.54(d, 1H, CH); 7.68(s, 1H, |
| | | | | CH); 9.16(s, 1H, CH); 12.12(br s, 1H, |
| | | | | NH exchanges with D₂O) |
| Ib | 75.75% | 287-289° C. | 1620(CN) | (DMSO) 2.55(s, 3H, CH₃); 3.67(s, 2H, |
| (X = CH₂, Y = H, | | (EtOH) | | CH₂), 7.10(t, 1H, CH); 7.26(t, 1H, |
| R = H, R₂ = CH₃) | | | | CH); 7.45-7.50(d, 3H, CHx3); 11.83 |
| | | | | (br s, 1H, NH exchanges with D₂O) |
| Ic | 82.14% | 350° C. | 1610(CN) | (CF₃COOD) 3.82(s, 2H, CH₂); 7.20-7.40 |
| (X = CH₂, Y = H, | | (dec.) | | (m, 2H, CHx2); 7.52(t, 2H, |
| R = H, R₂ = Ph) | | | | CHx2); 7.68-7.96(m, 4H, CHx4); 8.16-8.20 |
| | | | | (d, 2H, CH₂); 11.60(br s, 1H, NH |
| | | | | exchanges with D₂O) |
| Id | 78.66% | 175-178° C. | 1610(CN) | (CDCl₃) 3.74(s, 2H, CH₂); 3.96(s, 3H, |
| (X = CH₂, Y = H, | | | | CH₃); 7.20(t, 1H, CH); 7.32(s, 1H, |
| R = CH₃, R₂ = H) | | | | CH); 3.74(s, 1H, CH); 7.47(d, 1H, |
| | | | | CH); 7.53(d, 1H, CH); 8.83(s, 1H, CH) |
| Ie | 93.33% | 198-200° C. | 1600(CN), | (CDCl₃ + DMSO) 2.84(t, 2H, CH₂); |
| (X = CH₂CH₂, | | | 3170(NH) | 2.91(t, 2H, CH₂); 2.91(t, 2H, CH₂); |
| Y = H, R = H, R₂ = H) | | | | 7.26-7.38(m, 3H, CHx3); 7.45(s, 1H, |
| | | | | CH); 7.57(d, 1H, CH); 8.56(s, 1H, |
| | | | | CH); 11.18(br s, 1H, NH exchanges |
| | | | | with D₂O) |
| If | 96.25% | 201-203° C. | 1620(CN), | (CDCl₃ + DMSO) 2.57(s, 3H, CH₃); |
| (X = CH₂CH₂, | | | 3250(NH) | 2.96(t, 2H, CH₂); 3.05(t, 2H, CH₂); |
| Y = H, R = H, R₂ = CH₃) | | | | 7.08-7.24(m, 3H, CHx3); 7.35-7.45(m, |
| | | | | 2H, CHx2); 10.95(br s, 1H, NH |
| | | | | exchanges with D₂O) |
| Ig | 94.73% | 297-298° C. | 1640(CN), | (CDCl₃ + DMSO) 3.01(t, 2H, CH₂); |
| (X = CH₂CH₂, | | | 3170(NH) | 3.13(t, 2H, CH₂); 7.09-7.25(m, 3H, |
| Y = H, R = H, R₂ = Ph) | | | | CHx3); 7.49-7.57(m, 5H, CHx5); 8.06-8.11 |
| | | | | 8.11(m, 2H, CHx2); 11.56(br s, 1H, |
| | | | | NH exchanges with D₂O) |
| Ih | 63.63% | 277-278° C. | 1615(CN), | (CDCl₃ + DMSO) 7.41-7.55(m, 3H, |
| (CH═CH, Y = H, | | | 3180(NH) | CHx3); 7.62(d, 1H, CH); 7.88(d, 1H, |
| R = H, R₂ = H) | | | | CH, J_{AB}=6.4Hz); 8.23(d, 1H, CH); |
| | | | | 8.30(d, 1H, CH, J_{AB}=6.4Hz); 8.44(s, |
| | | | | 1H, CH); 12.10(br s, 1H, NH |
| | | | | exchanges with D₂O) |
| Ii | 90.90% | 326-328 C. | 1620(CN) | (CDCl₃ + DMSO) 2.64(s, 3H, CH₃); |
| (CH═CH, Y = H, | | | | 7.44(s, 1H, CH); 7.45-7.61(m, 2H, |
| R = H, R₂ = CH₃) | | | | CHx2); 7.67(d, 1H, CH, J_{AB}=8.8Hz); |
| | | | | 7.93(d, 1H, CH); 8.32(d, 1H, CH, J_{AB}=8.8Hz); |
| | | | | 8.38(d, 1H, CH); 12.03(br s, |
| | | | | 1H, NH exchanges with D₂O) |
| Il | 37.50% | 328-329° C. | 1630(CN), | (CDCl₃ + DMSO) 7.48-7.61(m, 5H, |
| (CH═CH, Y = H, | | | 3160(NH) | CHx5); 7.62(s, 1H, CH); 7.68(d, 1H, |
| R = H, R₂ = Ph) | | | | CH, J_{AB}=8.8Hz); 7.96(d, 1H, CH); |
| | | | | 8.11(d, 1H, CH); 8.15(d, 1H), CH); |
| | | | | 8.26(d, 1H, CH, J_{AB}=8.8Hz); 8.44(d, |
| | | | | 1H, CH); 13.42(br s, 1H, NH |
| | | | | exchanges with D₂O) |
| Im | 86.67% | 178-180° C. | 1620(CN) | (CDCl₃) 4.34(s, 3H, CH₃); 7.48-7.63(q, |
| (CH═CH, Y = H, | | | | 2H, CHx2); 7.70(d, 1H, CH, J_{AB}=8.4Hz); |
| R = CH₃, R₂ = H) | | | | 8.02(d, 1H, CH), 8.38(d, 1H, CH, |
| | | | | J_{AB}=8.4Hz); 8.40-8.47(m, 3H, CHx3) |

Pharmacological Section

Biological assays were carried out at the National Cancer Institute according to known experimental protocols [(a) Monks A., Scudiero D., Skehan P., Shoemaker R., Paull K., Vistica D., Hose C., Langley J., Cronise P., Vaigro-Wolff A., Gray-Goodrich M., Campbell H., Mayo J., Boyd M., J. Natl. Cancer Inst., 83, 757-766 (1991). (b) Paull K. D., Shoemaker R. H., Hods L., Monks A., Scudiero D. A., Rubinstein L., Plowman J., Boyd M. R., J. Natl. Cancer Inst., 81, 1088-1092 (1989). (c) Boyd M. R., Paull K. D., Rubinstein L. R., Cytotoxic Anticancer Drugs: Models and Concept for Drug discovery, Valeriote F. A., Corbett T., Baker L., Eds., Kluwer Academic Publishers: Amsterdam, 1992, pp. 11].

Table 1 reports the GI₅₀, namely the mean concentration (μM) of substance which causes a 50% inhibition on the cell growth in preliminary tests carried out on three cell lines (MCF7 breast, NCI-H460 microcitoma and SF-268 SNC).

Table 2 shows the GI₅₀ on various tumor cells.

In both cases compounds (Ic) and (Il) have shown significant antiproliferative activity.

TABLE 6

| | Ia | Ib | Ic | Ie | If | Ig | Ih | Il | Im |
|---|---|---|---|---|---|---|---|---|---|
| R₂ | H | CH₃ | C₆H₆ | H | CH₃ | C₆H₆ | H | C₆H₅ | H |
| X | CH₂ | CH₂ | CH₂ | (CH₂)₂ | (CH₂)₂ | (CH₂)₂ | CH═CH | CH═CH | CH═CH |
| R | H | H | H | H | H | H | H | H | CH₃ |
| GI₅₀ μM) | 74.1 | 75.8 | 2.9 | 26.6 | N.A. | N.A. | 35.4 | 1.28 | 33.1 |
| Mean | | | | | | | | | |

NA = products inactive in the preliminary tests on three cell lines (MCF7 breast, NCI-H460 microcitoma and SF-268 SNC).

TABLE 7

| | Ia | Ib | Ic | Ie | If | Ig | Ih | Il | Im |
|---|---|---|---|---|---|---|---|---|---|
| | GI₅₀ | GI₅₀ | GI₅₀ | GI₅₀ | GI₅₀ | GI₅₀ | GI₅₀ | GI₅₀ | GI₅₀ |
| Cell line | (μM) | (μM) | (μM) | (μM) | (μM) | (μM) | (μM) | (μM) | (μM) |
| Leukemia | 79.7 | 81.3 | 1.82 | 26.3 | N.A. | N.A. | 44.6 | 0.64 | 54.9 |
| Microcitoma | 57.8 | 93.3 | 3.39 | 21.8 | N.A. | N.A. | 22.3 | 2.36 | 17.7 |
| Colon | 81.2 | 100.0 | 1.00 | 25.7 | N.A. | N.A. | 33.8 | 0.70 | 38.9 |
| SNC | 86.7 | 75.8 | 2.95 | 28.2 | N.A. | N.A. | 32.3 | 1.21 | 23.9 |
| Melanoma | 77.3 | 79.4 | 2.34 | 28.8 | N.A. | N.A. | 33.8 | 2.71 | 43.6 |
| Ovary | 81.2 | 30.2 | 4.57 | 28.2 | N.A. | N.A. | 38.9 | 1.70 | 41.6 |
| Kidney | 67.3 | 91.2 | 6.31 | 27.5 | N.A. | N.A. | 38.0 | 2.04 | 20.8 |
| Prostate | 100 | 100.0 | 4.78 | 32.3 | N.A. | N.A. | 62.3 | 1.28 | 54.9 |
| Breast | 73.2 | 77.6 | 3.16 | 27.5 | N.A. | N.A. | 40.0 | 0.60 | 44.6 |
| Mean | 74.1 | 75.8 | 2.9 | 26.3 | N.A. | N.A. | 35.4 | 1.28 | 33.1 |

NA = inactive products.

1. Compounds of general formula (I) in which: 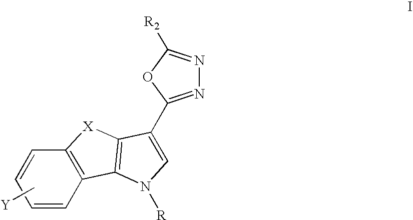
in which:
X is a CH₂, CH₂CH₂ or CH═CH group;
R is hydrogen, straight or branched C₁-C₄ alkyl, or phenyl-C₁-C₂-alkyl;
R₂ is hydrogen, straight or branched C₁-C₄ alkyl, or phenyl, optionally substituted with one more groups, which can be the same or different, selected from halogen (fluorine, chlorine, bromine, iodine), straight or branched C₁-C₄ alkyl, C₁-C₃ alkoxy, trifluoromethyl;
Y is hydrogen, halogen (fluorine, chlorine, bromine, iodine), straight or branched C₁-C₄ alkyl, C₁-C₃ alkoxy, trifluoromethyl.

2. Compounds as claimed in claim 1 wherein C₁-C₄ alkyl is methyl or ethyl; phenyl-C₁-C₂-alkyl is benzyl, optionally substituted on the phenyl ring with one more groups, which can be the same or different, selected from halogen (fluorine, chlorine, bromine, iodine), straight or branched C₁-C₄ alkyl, C₁-C₃ alkoxy, trifluoromethyl; alkoxy-C₁-C₃ is methoxy or ethoxy.

3. Compounds as claimed in claims 1 or 2 claim 1 in which X is a CH₂ or CH═CH group, R is hydrogen or methyl and R₂ is hydrogen, methyl or optionally substituted phenyl, as defined in claim 1.

4. A compound as claimed in claim 1 selected from:
3-[[1,3,4]oxadiazol-2′-yl]-1,4-dihydro-indeno[1,2-b]pyrrole;
3-[[1,3,4]oxadiazole-5′-methyl-2′-yl]-1,4-dihydro-indeno[1,2-b]pyrrole;
3-[[1,3,4]oxadiazole-5′-phenyl-2′-yl]-1,4-dihydro-indeno[1,2-b]pyrrole;
1-methyl-3-[[1,3,4]oxadiazol-2′-yl]-1,4-dihydro-indeno[1,2-b]pyrrole;
3-[[1,3,4]oxadiazol-2′-yl]-4,5-dihydro-1H-benzo[g]indole;
3-[[1,3,4]oxadiazole-5′-methyl-2′-yl]-4,5-dihydro-1H-benzo[g]indole;
3-[[1,3,4]oxadiazole-5′-phenyl-2′-yl]-4,5-dihydro-1H-benzo[g]indole;
1-methyl-3-[[1,3,4]oxadiazol-2′-yl]-1H-benzo[g]indole;
3-[[1,3,4]oxadiazol-2′-yl]-1H-benzo[g]indole;
3-[[1,3,4]oxadiazole-5′-methyl-2′-yl]-1H-benzo[g]indole;
3-[[1,3,4]oxadiazole-5′-phenyl-2′-yl]-1H-benzo[g]indole.

5. A compound of formula (II) in which X, Y and R are as defined in claim 1.

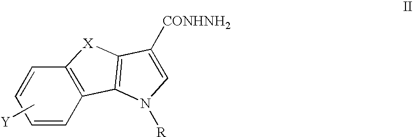

6. Compounds of claim 1 as medicaments.

7. A method of treating tumors, comprising administering an effective amount of a compound according to claim 1.

8. The method as claimed in claim 7 in which tumors are: leukemias, colon tumors, breast tumors.

9. Pharmaceutical compositions containing the compounds of claim 1 in admixture with suitable excipients and/or carriers.
